(19) United States
(12) Patent Application Publication
(10) Pub. No.: US 2022/0000711 A1
(43) Pub. Date: Jan. 6, 2022

(54) ACUPRESSURE MEDALLION, METHOD AND DEVICE FOR ITS MANUFACTURE AND METHOD OF REDUCTION OF PAIN FROM ACUPRESSURE REFLEXOTHERAPY

(71) Applicants: Roman BURKO, Vinnitsa (UA); Tymur BURKO, Vinnitsa (UA)

(72) Inventors: Roman BURKO, Vinnitsa (UA); Tymur BURKO, Vinnitsa (UA)

(21) Appl. No.: 17/066,082

(22) Filed: Oct. 8, 2020

(30) Foreign Application Priority Data
Jul. 6, 2020 (UA) A202004052

(51) Int. Cl. A61H 39/04; A61L 31/00; A61L 31/16; A61L 31/02; A61L 31/04; B29C 39/02; B29C 39/26

(52) U.S. Cl. CPC A61H 39/04; A61L 31/005; A61L 31/16; A61H 2201/1695; A61L 31/049; B29C 39/026; B29C 39/26; A61L 31/026

(57) ABSTRACT

The acupressure element includes a base with spiked applicators and an additional layer of low-melting substance, which creates a surface flush with the points of the spiked applicators. The layer hides the points and prevents pain. During a reflexotherapy procedure, the layer of low-melting material melts, either under the influence of human body heat or with the use of external heating. The points of the spiked applicators are gradually released and begin to press on the skin. The pressure on the skin does not occur immediately, allowing to prepare for increased pain later and acclimate accordingly.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The invention relates to medicine, namely to devices for massage, acupressure or reflexotherapy, which are used to treat and prevent various diseases, in particular for acupressure to tone up blood circulation, for relaxation, disease prevention and strengthening the body's immunity.

### 2. Description of Related Art Including Information Disclosed Under 37 CFR 1.97 and 37 CFR 1.98

The use of acupressure methods, which are usually combined under the name of reflexotherapy, is known in the field of medicine. It is most often understood as, in particular, pressing on individual points on human skin, the so-called reflex points, which causes normalization or improvement of the body's functioning. Also, such methods are often referred to as types of massage or self-massage. There are many claims about their effectiveness in a variety of conditions and diseases, from high blood pressure to cancer, reflexotherapy is also used in sports medicine. Due to relative simplicity and safety of use, reflexotherapy is used without prescription, as desired.

One of the most famous devices for acupressure reflexotherapy is the Kuznetsov applicator, which gave rise to a large family of similar applicators, its different variations. Structurally, these masseurs are similar and have the form of flat elements of various shapes, from which protrude moderately sharp outgrowths, like spikes or needles. Examples of such devices and methods of their application are described in the following patent documents: Russian patents 2017478, 2045946, 2034526, 2133602 and 2145834, etc.; Ukrainian utility models 72870, 76348, 89930, 112193, 130803, etc.

Such applicators or applicator elements are usually fixed on substrates with needles on the outside for use in a similar method for all of them—a person lies on an applicator, or in some other way presses, or fixes an applicator on the skin, with pressure applied, if effect is desired on such portions of skin that are impractical to lie down on. Accordingly, the number of elements and/or applicators area, or the number of spikes or needles, should be such that body weight or pressure force exerted on the skin does not cause skin piercing and injury. However, developers of applicators avoid too many needles or spikes, citing disappearance of massaging effect. Therefore, acupressure reflexotherapy with the help of applicators is usually associated with pain, which discourages many potential users, not allowing them to take advantage of it and feel its positive effect. None of the known technical solutions in this field offers possibilities for adaptation, hardening or acclimation of prospective patients to pain during acupressure with applicators.

### BRIEF SUMMARY OF THE INVENTION

### Technical Problem

The present invention is aimed at solving the problem of pain during acupressure massage, or acupressure reflexotherapy with applicators. At least at the initial stages of reflexotherapy it is necessary to reduce the pain of acupressure, so that the patient can get used to or acclimate to unpleasant sensations, adapt to massage with applicators.

### Solution of the Problem

The task is solved by improving the applicator or acupressure elements by providing an additional layer of low-melting substance, which creates a surface flush with the tips of the spiked reflex action outgrowths, thus hiding these points and preventing pain. During massage or reflexotherapy procedure, this layer of low-melting material melts, either under the influence of human body heat or with the use of external heating, and the tips of the spiked reflex action outgrowths are gradually released and begin to press on the skin. The fact that the pressure on the skin with the tips of the spiked outgrowths does not occur immediately, allows the patient to prepare for increased pain later and acclimate themselves.

### Useful Results

Thus, present invention provides for the first time a possibility to reduce pain during reflexotherapy with acupressure applicators by hiding the spiked reflex effect outgrowths in the built-up layer of low-melting material. During acupressure reflex massage low-melting material melts (with the help of human body heat or with an external heat source) and gradually releases the tip of the spiked outgrowths, providing slow dynamics of unpleasant sensations when the spiked outgrowths press on the skin. Additionally, the possibility is provided to combine massage with action of low-melting material, if it a) melts at a lower or a higher temperature relative to skin temperature, and b) if the low-melting material contains additional excipients and/or active ingredients for medicinal, cosmetic or consumer purposes. Acupressure applicators or acupressure elements that were improved in accordance with the present invention are also called “acupressure medallions”.

### BRIEF DESCRIPTION OF THE SEVERAL VIEWS OF THE DRAWINGS

FIG. 1 is a side schematic view of an acupressure element (medallion).

FIG. 2 is another side schematic view of an acupressure element (medallion).

FIG. 3 is a side schematic view of an embodiment of the device for the manufacture of the acupressure element, showing one cell of the device assembled with a solid part of the acupressure element, wherein the low-melting material is not shown.

FIG. 4 is a side schematic view of an embodiment of the device for the manufacture of acupressure element, showing one cell of the device assembled with a solid part of the acupressure element, wherein the low-melting material is not shown.

FIG. 5 a side schematic view of another embodiment of the device for the manufacture of acupressure element, showing one cell of the device assembled with a solid part of the acupressure element, wherein the low-melting material is not shown.

FIG. 6 a side schematic view of another embodiment of the device according to the invention, wherein the cells have a removable bottom, showing one cell of the device assembled with a solid part of the acupressure element, wherein the low-melting material is not shown.

FIG. 7 is a side schematic view of an embodiment of the device according to the invention, wherein the cells have a bottom made together with the walls, showing one cell of the device assembled with a solid part of the acupressure element, wherein the low-melting material is not shown.

FIG. 8 a side schematic view of an embodiment of FIG. 7, showing a different orientation of the solid part.

FIG. 9 is a side schematic view of an embodiment of the device according to the invention with “free” cells, showing one cell of the device assembled with a solid part of the acupressure element, wherein the low-melting material is not shown.

FIG. 10 is a perspective view of a depiction of one of the embodiments of the device (free cells, hexagonal shape of the applicator elements).

FIG. 11 is a set of photographs, depicting various variants of applicator elements suitable for use in the method according to the invention.

### LEGEND

1. the applicator element, or the solid part of the acupressure element according to the invention;
2. a low-melting material;
3. the base of the solid part;
4. the supporting side of the base;
5. the working side of the base;
6. spiked reflex action outgrowth;
7. surface of the low-melting material layer;
8. tip (point) of a spiked outgrowth;
9. the cell wall of the device according to the invention;
10. the edge of the solid part's base of an acupressure element;
11. removable bottom (cover, base) of a cell;
12. a cell made integrally with the bottom, close contact;
13. “free” cell, i.e. made integrally with the bottom, without close contact.

### DETAILED DESCRIPTION OF THE INVENTION

This section describes in detail possible embodiments of the present invention.

One of the main aspects of the present invention is an acupressure element. In preferred embodiments of the present invention, the acupressure element comprises a) a solid part, which includes a base having support and working sides and on the working side there is at least one reflex action spiked outgrowth, and b) a layer of low-melting material capable of melting or softening at a temperature that human skin can withstand at least 1 minute, and the said layer of low-melting material is made by filling the volume on the working side of the base, between the base and the spiked outgrowth (outgrowths) so that said layer forms a surface that is essentially at the same level with a point(s) of spiked outgrowth or outgrowths, or at a level lower than the said point (points) by 1-5 mm. In part of the embodiments of the present invention, said surface of the low-melting material layer is essentially at the same level as the tip of the spiked outgrowth or outgrowths. In other embodiments of the present invention, said surface of the low-melting material layer is lower than said tips by 0.5-5 mm.

In most preferred embodiments, the mentioned above element has low-melting material that melts or softens at temperatures from 0 to 48° C.

In the majority of preferred embodiments, the low-melting material may be selected from a group comprising ice and an aqueous gel, which may be selected from a gel, based, for example, on agarose, agar-agar, carrageenan, alginates and gelatin.

In other embodiments of the present invention, the low-melting material may be selected from a group comprising, for example, solid vegetable or animal fat, wax, propolis, vaseline, paraffin, a mixture of paraffin and ozokerite, or a mixture of any or all of these materials.

In some embodiments of the present invention, the low-melting material may further contain excipients and/or active ingredients for medicinal, cosmetic or consumer purposes. These may include extracts of medicinal plants, teas, infusions, oils etc. Such plants can be, for example, aloe vera, lemon balm, mint, celandine, matcha tea, rose, lavender, and cucumber. Sources of such supplements can also be honey, shilajit etc.

In particular embodiments of the present invention, the low-melting material is gallium or its low-melting alloy. Among the low-melting alloys of gallium may be its alloys with indium, tin, antimony, bismuth etc.

In preferred embodiments of the present invention, the base and/or spiked outgrowths of the above mentioned acupressure element may be made of a material selected from the group containing: plastic (polyethylene, polypropylene, Teflon, polystyrene), wood, metal, rubber, silicone and ceramics.

In the most typical embodiments, the base of the acupressure element has the shape of a square, circle, oval, hexagon, triangle or polygon.

In many of the most preferred embodiments, the spiked reflex action outgrowths of the acupressure element have the shape of a cone, a pyramid with a base having three-, four- and/or more angles, a petal or blade with a tapering angle or side, a nail or a needle.

Also in preferred embodiments of the present invention, the support side of the acupressure element base can be made with means for fixing or fastening on an underlying surface, substrate or mat. In other embodiments, the acupressure element may be non-attachable to a substrate.

In further embodiments, the above mentioned fastening means may be represented by one or more of the following: a hole or holes, a loop, a button, a hook and/or a sticky fastener (Velcro™).

Another important aspect of the present invention is a device for manufacturing the acupressure element described above.

In one of the preferred embodiments of the invention, the device for manufacturing an acupressure element is a bottomless cell, or a plurality of bottomless cells connected side by side, where said cell has a shape and size sufficient to a) accommodate one base of the acupressure element being tightly adjacent to its edges, and the height of the cell is not less than 0.5-5 mm of the sum of the height of the said action spiked outgrowth(s) and the thickness of the said base of the acupressure element, or the said cell has a shape and size sufficient to b) accommodate all spiny outgrowths of one acupressure element with a close fit to the working side of its base, and the height of the cell is not lower than 0.5-5 mm of the height of the spiked outgrowth(s) over the said base of the element.

By close fitting or tightly adjacent it should be understood that the edges of the walls of the cell or cells of the said device are made so as to ensure the tightness of the contact of the cell with the base of the acupressure element.

By tightness it is meant that when the horizontally placed base of the acupressure element is adjacent to the cell walls and the molten low-melting material specified above is poured into the formed container, the molten material will not leak at the point of contact of the acupressure elements base with the cell walls.

In a further embodiment of the invention, said cell or cells of the device are shaped and sized to accommodate the base of an acupressure element with a close fit to its edges, the height of the cell being not lower than 5 mm then the sum of the height of said spiked outgrowth and thickness of said base of the acupressure element. In a further embodiment of the invention, the height of the cell is 0.5-5 mm lower than the sum of the height of the said spiked reflex action outgrowth (outgrowths) and the thickness of the said acupressure elements base. In another separate embodiment of the invention, the height of the cell is equal to or greater than the sum of the height of the said spiked reflex action outgrowth (outgrowths) and the thickness of the said acupressure elements base.

In another part of the embodiments of the invention, said cell has the shape and dimensions to accommodate all the spiked outgrowths of one acupressure element with a close fit to the working side of the base of its solid part, and the cell height is not 5 mm lower than the height of spiked reflex action outgrowth (outgrowths) above the said base of the element. In one of the further embodiments of the invention, the height of the cell is 0.5-5 mm lower than the height of the spiked reflex action outgrowth (outgrowths) over the said base of the element. In yet another further embodiment of the invention, the height of the cell is equal to or greater than the height of the spiked reflex action outgrowth (outgrowths) over the specified base of the element.

In some embodiments of this aspect of the invention, the device for manufacturing the acupressure element further includes a bottom as a separate part, preferably flat, made with means for close fitting to the edges of the said cell or cells.

By close fitting it is understood that the edges of the walls of the cell or cells of the above device are made so as to ensure the tightness of the contact of the cell with the said flat bottom.

By tightness is meant that when the horizontally placed flat bottom is adjacent to the cell walls and the so formed container is filled with molten low-melting material specified above, the molten material will not leak at the point of contact of the flat bottom with the cell walls. Moreover, if a solid part of the acupressure element is placed with the working side of the base, i.e. outgrowths, inward, in the so formed container after pouring the low-melting material in it, and the support side of the base of the acupressure element turned down, the molten material will not leak at the base of the acupressure element.

In a further embodiment of the invention, said bottom is formed integrally with said cell or cells.

In another group of preferred embodiments of this aspect of the invention, such a device includes one cell with a bottom, or a plurality of cells with a bottom, connected side by side, and the size and shape of the cell are sufficient to accommodate a solid part of the acupressure element, freely, without contact between the edge of the base of the solid part of the acupressure element and the cell walls; moreover, the height of the cell is not lower than 5 mm of the height of the spiked reflex action outgrowth(s) over the above mentioned base of the element. In one further embodiment of the invention, said free cell, or cells, with bottom, of the device for manufacturing the acupressure element, has (have) a shape corresponding to the shape of the base of the acupressure element, but without making close contact.

In certain embodiments of this aspect of the invention, said cells have a height that is lower than 0.5-5 mm of the sum of the height of the above-mentioned spike reflex action outgrowth(s) and the thickness of the above mentioned base of the acupressure element.

In other embodiments of this aspect of the invention, said cells have a height equal to or greater than the sum of the height of the above spiked outgrowth (outgrowths) of reflex action and the thickness of the above base of the acupressure element.

In the most preferred embodiments of this aspect of the invention, the above mentioned device may be made of a material selected from a group consisting of, for example: rubber, silicone, metal, plastic, cardboard, paper, foil, foil or plastic laminated paper and wood. In some embodiments of the invention, the above mentioned device may be designed for single use.

Another important aspect of the present invention is a set that includes at least one acupressure element according to the invention and a device for its manufacture with at least one cell. In a preferred embodiment, the acupressure element or elements in said set according to the invention is placed (are placed) in said cell or cells. In a separate embodiment of this aspect of the present invention, said kit further comprises an outer package, in particular a package that prevents the separation of the acupressure element from the device. In another separate embodiment of this aspect of the present invention, the package is made to prevent leakage or separation of the low-melting material from the base of the acupressure element during temperature changes when transported and stored, or the above mentioned device is made so that together with the above mentioned element placed in it, it will prevent separation of acupressure element from the device and/or prevent leakage or separation of low-melting material from the base of the acupressure element during temperature changes when transported and stored.

Another important aspect of the present invention is a method of manufacturing the above mentioned acupressure element. In the most preferred embodiments, this method includes the steps of:

a) heating the above mentioned low-melting material to a liquid or softened state; further, either

b1) covering the working side of at least one base of the solid part of the acupressure element placed horizontally with the working side up, with the device that includes a cell(s) or bottomless cell(s), or placing the solid part of the acupressure element base down in the cell of the device, that has a removable bottom or cell, that is made integral with the bottom, and adding the required amount of liquid or softened low-melting material to the said cell(s) with a solid part of the acupressure element at the bottom, or

b2) adding the required amount of liquid or softened low-melting material to at least one cell of the device having a removable bottom, or to a cell made integral with the bottom, and placing in such a cell with low-melting material a solid part of the acupressure element, base up, and

c) cooling the device together with its contents until the low-melting material acquires acceptable hardness, and then

d) removing the manufactured acupressure element for use or storage and, if necessary, repeating all stages, starting with a).

In a separate embodiment of this method, the manufactured acupressure element in stage d) is stored until use without removing from the device.

In part of preferred embodiments of this aspect of the invention an amount of low-melting material is added, sufficient to create a layer of low-melting material with a surface flush with the height of the specified spiked reflex action outgrowth(s).

In another part of preferred embodiments of the present invention an amount of refractory material is added, sufficient to create a layer of low-melting material with a surface below 0.5-5 mm of the height of the spiked reflex action outgrowth(s). When using in this embodiment of a method a device comprising a cell or cells with a bottom and close contact, and a variant of placing the solid base with outgrowths inwards, the device with low-melting material and the solid base inside, is turned over with the support side of the solid base down, to settle the low-melting material.

The most important aspect of the present invention is a method of reducing pain during acupressure reflexotherapy. In preferred embodiments of this aspect of the invention:

a) at least one acupressure element described above, manufactured in accordance with the method described above, is laid down on a surface with the support side of the base down,

b) pressure is provided on it by a part of the skin surface of the human body selected for reflexotherapy, until the appearance of pain, and

c) stages a) and b) are repeated after a reasonable period of time, to reduce pain or to get used to it and to get a possibility of continuing the procedure of acupressure reflexotherapy for a longer period of time.

In a further embodiment of this aspect of the invention, after reducing pain or becoming accustomed to an acupressure element having a surface of a layer of fusible material that is substantially flush with the tip(s) of a spike or outgrowth, repeat all steps with the said element having the surface of low-melting material layer, which is already at a level that is lower than the said tip(s) by 0.5-5 mm.

In some particular embodiments of this method, at least one acupressure element at the skin pressure stage is additionally heated on the support side of the base to 48° C.

In other embodiments of this method, a substrate or mat is additionally used to lay down on it at least one acupressure element, and the said at least one element is fixed thereto by the above-mentioned fixing or fastening means in accordance with the knowledge of those skilled in the art. Said substrate or a mat may be made of fabric, rubber, silicone etc.

In preferred embodiments of the method of reducing pain during acupressure reflexotherapy, according to the present invention, the pressure is provided by a person that lies on the acupressure element or elements.

In some other embodiments, the pressure is provided by fixing the acupressure element(s) to human skin by means of bandages, tapes, straps or fasteners, which are provided with the substrate.]

### INDUSTRIAL APPLICABILITY

This section deals with the production and use of various embodiments of the present invention.

The present invention improves acupressure applicators or acupressure elements of applicators for acupressure applicator reflexotherapy (massage), which originate from the famous Kuznetsov applicator, making it possible to apply this procedure to persons who could not use it in the previous, classical performance. Typically, such acupressure applicators are used for treatment and prevention of various diseases, in particular, for acupressure of the body in order to tonically improve blood circulation, relaxation, disease prevention and strengthen the body's immunity. Conventional, classic applicators consist of many applicator elements of arbitrary shape (square, round, etc.), equipped with several (or many) spike-formed outgrowths. In many of the most preferred embodiments, the spiked reflex action outgrowths of the acupressure element have the shape of a cone, a pyramid with a base having three-, four- and/or more angles, a petal or blade with a tapering angle or side, a nail or a needle.

Most often, acupressure applicators are used when fixed on a pad or a mat, on which a person in need of this reflexotherapy acupressure procedure, lies on the bare skin of the torso, often the back. It is possible to apply acupressure applicators to other parts of the human skin, provided that the pressure on a single acupressure element is comparable with the pressure exerted by the human body under its own weight on a separate acupressure element. It is known that the higher the density of spiked outgrowths—the lower the pain or discomfort, but the less the effect of reflexotherapy or massage. Accordingly, to increase the effect of the massage, it is necessary to use a less frequent arrangement of acupressure elements and a less dense arrangement of spiked outgrowths on them. Location of acupressure elements of applicators, their shape and location of spiked reflex action outgrowths on them can be any acceptable in this field, because it does not relate to the essence of the present invention. The essence of the present invention is that it reduces pain or discomfort, which makes it impossible to use applicator acupressure reflexotherapy for a significant swathe of people who could benefit from this procedure. Thus, lowering the pain threshold of acupressure allows this procedure to be performed by those who were not previously able to do so.

For example, if we assume that the spiked outgrowths of acupressure applicators when used can due to the elasticity of the skin sink up to 3-5 mm without damaging it, then, respectively, applicators with infrequent location of spiked elements may have them not higher than 3-5 mm to protect the user from skin damage. Applicators that will have frequently located spiked outgrowths, for example, in the form of needles, may have a height of these growths over 3-5 mm, if their total number is sufficient to support the weight of the user's body (or sufficient to withstand pressure on acupressure applicators attached to the skin) so that the spiked outgrowths when using applicators do not damage the skin.

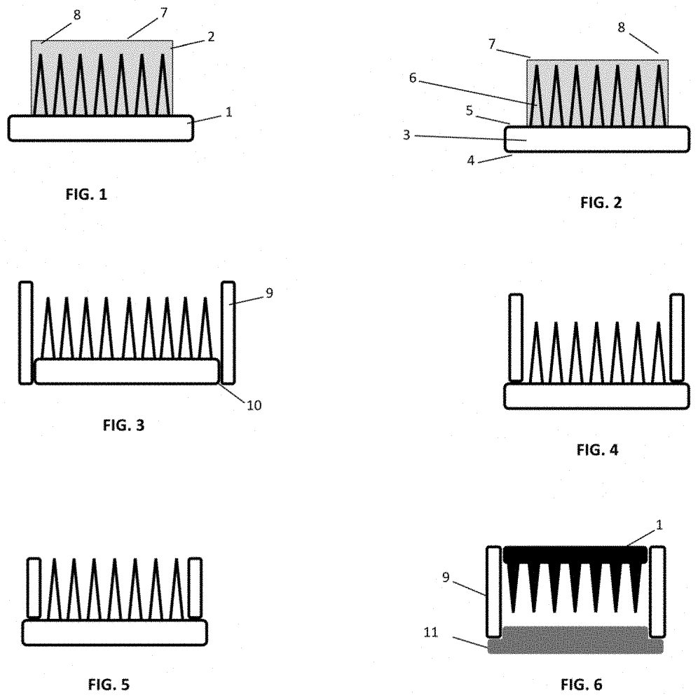

The purpose of the present invention is achieved by creating and providing for acupressure reflexotherapy or massage new acupressure elements, so-called acupressure medallions, as, for example, shown in FIGS. 1 and 2. The acupressure element of the present invention, or the acupressure medallion, as shown in FIG. 1, includes a classic applicator element, which comprises the solid part (1) of the acupressure element according to the invention, and a layer of low-melting material (2). The solid part (1) of the acupressure element, as shown in FIG. 2 consists of a base (3) having support (4) and working (5) sides; on the working (5) side there is at least one, often more, spiked outgrowth of reflex action (6). The layer of low-melting material (2) of the acupressure element according to the invention is usually fused on or formed on the working side of the base (3), i.e., on the side of the spiked outgrowth (6) or outgrowths. According to the present invention, there are two approaches to its embodiment: the first is the formation of a low-melting layer so that it creates a flat surface (7) flush with the tip(s) or point(s) (8) of the spiked outgrowths (6), as in FIG. 1, the second is the formation of a surface (7) of the low-melting layer 0.5 . . . 5 mm below the tips (8) of the spiked outgrowths (6), as in FIG. 2.

Thus, instead of lying down (pressing other areas of skin) on the usual elements of applicators with uncovered tips of spiked outgrowths, which causes discomfort, it is possible to start the procedure with a comfortable feeling, lying down (applying to the skin) smooth acupressure medallions.

As a low-melting layer any material can be used that meets hygienic requirements for a material that can be applied to human skin, and which has a melting or softening temperature that a person can withstand without physical harm or injury (burns, hypothermia) of the skin.

The melting or softening of the layer of low-melting material, which begins immediately after the start of exposure of this material to an appropriate temperature for the material, releases the tip of the spiked outgrowths and allows the person, undergoing reflexotherapy or massage, to begin gradually feeling the pressure of spiked outgrowths. Gradual increase of unpleasant sensations gives a chance for such person to prolong this procedure and to start to get used, to adapt to pain or discomfort.

Depending on the degree of adaptation of said person to the pain of spiked outgrowths, two approaches can be used to make acupressure medallions: a novice person will most likely need to start the reflexotherapy procedure with smooth medallions, where the surface of the layer of fusible material is level with the height of the spikes. When a person who wants to get used to acupressure reflexotherapy has already undergone some adaptation, acupressure medallions can be used, from which the spiked outgrowth tips protrude by 0.5-5 mm. According to experience, individuals adapted to acupressure elements with a length of spiked protrusions up to 5 mm do not require further adaptation, and can proceed directly to conventional applicators.

Acupressure medallions can be used even if the layer of low-melting material is thicker than the height of the spiked outgrowths, but the excess thickness of the low-melting material layer will not give additional effect of the invention, it will only prolong the time required for initial melting or softening of low-melting material for the person undergoing the procedure, to begin to feel the tips of the spiked outgrowths.

As mentioned above, the limitations for a low-melting material are primarily hygienic. Among hygienically suitable materials are those that melt under exposure to human skin temperature (usually considered to be 36.6±0.5° C.). These include ice, gallium and its alloys (with indium, tin, antimony, bismuth, etc.), some solid fats (vegetable, animal or mixtures thereof), wax, propolis, Vaseline™, paraffin, a mixture of paraffin and ozokerite, or a mixture of any or all of these materials. An aqueous gel selected, for example, from a gel based on agarose, agar-agar, carrageenan, alginates and gelatin can also be used.

Materials, for which the heat of human skin is insufficient to melt or soften, external heat sources may be used, such as various heaters (water, electric, etc.). These can be materials based on paraffins, waxes, some gels.

It is known that a person can withstand on his skin materials of a limited temperature range. It is believed that temperatures in the range from 0 to 48° C. are safe, at least a person can withstand them for the time required to train endurance, getting used to the sharp spiked outgrowths and applicator elements.

Additionally, by maintaining temperatures significantly above (above 40° C.) or below (for example, 0° C.) the surface skin temperature, additional massage, tonic or other effects can be achieved due to the temperatures effect.

In addition, the low-melting material, with the exception of gallium and its alloys, may also contain excipients and/or active ingredients for medicinal, cosmetic or consumer purposes. These may include extracts of medicinal plants, teas, infusions, oils etc. Such plants can be, for example, aloe vera, lemon balm, mint, celandine, matcha tea, rose, lavender, and cucumber. Sources of such supplements can also be honey, shilajit etc. Accordingly, during the massage procedure with additional medicinal or cosmetic substances, the person gets an additional effect, which can even be medicinal. The use of additional medicinal, cosmetic or additional substances may be limited by allergies or an increased reaction to certain components.

Production of acupressure medallions is possible both separately, one after another, and together, according to a method of application. If the consumer lays down the medallions one after the other, then an acceptable method of manufacture would be a single manufacture. If a consumer plans to use medallions in an integrated manner, pre-fixed on a substrate (mat, etc.), as part of the finished applicator, etc., then the most convenient method of manufacture will be one that allows simultaneous production of the number of medallions used for the entire applicator together, i.e. the number of medallions required for one user to perform one procedure.

For the manufacture of acupressure medallions, a special device is used, disclosed in this invention. The first variant of a device for manufacturing of acupressure element is a bottomless cell or a set of bottomless cells connected side by side, having a shape corresponding to the shape of the solid part (base) of the acupressure element, which before pouring the molten low-melting material is put on the solid part acupressure element, located horizontally, with the support side down, and the cell walls provide close contact with the base of the solid part of the acupressure element. One embodiment of the device can be seen in FIG. 3. The tight fit of the cell walls (9) to the edges (10) of the base of the solid part ensures that the molten material does not spill through the cracks. If the structure of the solid part of the acupressure element allows close contact with the working side of its base around the spiked outgrowths, as in FIG. 4, such an option is also possible. In order to be able to pour the low-melting material to cover the points, the height of the cell walls must be at least flush with the points of the spiked outgrowths, or higher. In the case of making acupressure medallions for partially adapted consumers, the cell walls may be below the height of the spiked outgrowths by 0.5-5 mm, as illustrated in FIG. 5, but in this case such a device can be used only for the manufacturing of acupressure medallions, having 0.5-5 mm protruding points of spiked outgrowths.

The material for the device for the manufacture of acupressure element may be silicone, rubber, plastic (polyethylene, polypropylene, polystyrene, etc.), as well as metal, such as aluminum; also the material for the manufacture of acupressure element can be cardboard, paper, foil, paper laminated with foil or plastic, and wood. The device can be made of one material or a combination of materials, for example, with another material at the point of contact with the base of the acupressure element, to ensure a tight fit. The person skilled in the art should not have problems with choosing the most suitable material or combination of materials for the manufacture of the device according to the invention.

After hardening of the low-melting material, the finished acupressure element (medallion) can be removed, for example, by stretching the cell (if it is made of rubber or silicone), and by pushing it to one or both sides of the cell, depending on how it's walls are made at the point of contact with the base of the acupressure element.

The cell walls of the device can be made to contact the base of the acupressure element with a tight fit on one side only, or with the possibility of a tight fit on both sides of the cell, regardless of which side of the device cell is placed on the solid part of the acupressure element.

The finished acupressure medallion may need to be stored at a low temperature: in a refrigerator or cold chest, thermos or box prepared for cold storage (provided with elements for storage/accumulation of cold, etc.).

For convenience of use and distribution (transportation), the acupressure element or acupressure medallion may not be removed from the device for its manufacture after manufacture, and stored with it before use or transportation, thus forming a set of acupressure medallion and device for its manufacture. Said device may be designed to prevent spontaneous separation of the medallion from the device, to prevent leakage or separation of the low-melting material from the base of the acupressure element during temperature changes while transported and stored. Alternatively, said set may further comprise packaging that will actually prevent the separation of the acupressure element from the device and/or prevent leakage or separation of the low-melting material from the base of the acupressure element during temperature changes while transported and stored.

To produce individual acupressure medallions, it is sufficient to have separate cells that cover one element each. For the manufacture of acupressure medallions, which are already fixed in the applicator (on the substrate, mat, etc.), the device for the manufacture of acupressure elements must consist of such a number of cells connected together to ensure the simultaneous formation of the required number of acupressure medallions. Thus, the cells for forming a plurality of acupressure elements at once should be connected side by side in a continuous frame or shape, for example, on the principle of honeycombs, if the base of the solid part of the acupressure element has a hexagonal shape. If the solid base of the acupressure element has a different shape, the cells may not form “cells”, but be connected into a continuous system by means of additional structural elements. The number and relative position of the cells, if the device according to the invention has more than one, for the simultaneous manufacture of more than one acupressure medallion, may be arbitrary, but may be provided for the simultaneous manufacture of a number of acupressure medallions based on solid parts (applicator elements) pre-fixed on the substrate, mat, etc., for example, as part of the finished applicator. One skilled in the art will be able to select the desired method of connecting cells and, accordingly, the manufacture of a device that has more than one cell, without involving additional inventiveness. The cells can be fastened with separate parts, capable of assembly with other cells and disassembly, or in a non-detachable design. The cells, for example, may be attached to each other directly, or be part of a honeycomb structure, where the outer side of the wall of one cell is the inner side of the other. The cells can also be fastened at a distance from each other, using any suitable structural elements, forming a frame-like structure or a structure similar to a flat base, which can hold the cells both in the middle of the walls and closer to any of their edges, or directly joining one of the edges, or it can be simultaneously two flat bases, each connecting its own edge of the cells, and so on.

With a sufficient height of the walls of the cell(s) for the manufacture of acupressure medallions with different coating of spiked outgrowths can be adjusted by the amount of low-melting material poured into the cell or cells, in order to be able to produce acupressure medallions both fully coated with low-melting material and medallions, which have 0.5-5 mm protruding spiked outgrowths.

An embodiment of the device for manufacturing the acupressure element is shown on FIG. 6, there is a cell or cells that have a removable bottom (or base, or lid) (11) that is in close contact with the walls of the cell or several cells, close enough that poured into the cell, which stands on the base, molten fusible material does not spill through the cracks. If said device includes a plurality of cells, said removable bottom may belong as a separate part to each cell, or form an integral structure that forms a bottom simultaneously for all cells of this device. In this embodiment of the device (cell or cells with a removable bottom), the manufacture of the acupressure element can be performed differently: first, the cell standing on the bottom is filled with the required amount of molten low-melting material, then a solid part of the acupressure element is placed in, with the spiked outgrowths downwards, and waiting for cooling and solidification. If desired, the solid part can be placed in a cell with a removable bottom with the support side down, and then filled with molten low-melting material.

The requirements for the material from which the above mentioned removable bottom (or cover, or base) (11) is made are similar to those for the device according to the invention as a whole, mentioned above.

In a preliminary embodiment of the device, it may be provided with means or structural elements for fixing the cell(s) on the bottom or base, to increase the stability of the structure and prevent leakage of molten low-melting material. Specialists in the field will be able to develop means for fixing the cell walls on the said bottom, as based on variants of making cell walls and/or bottom, as well as with the use of various materials and their properties (elastic, resilient rubber, silicone rubber, etc.), or combinations of structural elements and properties of the material, because it has nothing to do with the essence of the present invention.

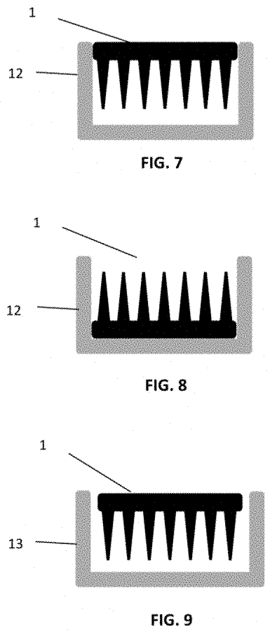

This variant can be modified so that the cells and the bottom are made as a whole, as illustrated in FIG. 7, as a cell together with the bottom (12), which is hermetically sealed by the base of the solid part (1) of the acupressure element, or more than one cell connected together side by side, according to the desired configuration of acupressure elements, which the specialist determines depending on needs or the presence of a ready-made applicator with a certain set of applicator elements. In this embodiment, it is possible to manufacture an acupressure medallion or medallions by immersing the solid part(s) of the acupressure element in a cell with pre-poured molten low-melting material. Also, due to the close contact between the cell walls and the bases of the acupressure elements, which prevents the molten material from pouring out, it is possible to turn the assembled device upside down after immersion so that solidification occurs when the device with the acupressure elements immersed in it lies horizontally on the supporting sides of the solid parts down. In this configuration, this version of the device will not completely fill the spiked outgrowths, leaving them sticking out 0.5-5 mm of height. This embodiment of the device according to the invention is even capable of being used in another embodiment, when the solid part of the acupressure element is placed in a cell with the bottom, supporting side of the base downwards, as shown in FIG. 8, and then pour the low-melting material.

A separate embodiment of the device for manufacturing acupressure elements illustrated in FIG. 9, the device comprises at least one “free” cell (13), i.e. a cell with a bottom, and the size and shape of the cell are sufficient to accommodate a solid part of the acupressure element, but freely, i.e. without close contact between the edge of the base of the solid part of the acupressure element and the cell walls. If it is necessary to manufacture many acupressure elements at once, such free cells are made joined together side by side, in such a configuration and at such a distance from each other as to satisfy a certain configuration of applicator elements fastened together on a substrate (mat, fabric, etc.), i.e., for the manufacture of acupressure medallions on a ready-made applicator. The means for fastening such cells can be different, as mentioned above for previous embodiments of the device according to the invention, i.e., a person skilled in the art will not have much difficulty choosing either the material or layout of cells or the option of combining them into one device for making acupressure elements with “free” cells. For example, such a device may include a flat base in which at least one recess is made, and the dimensions and shape of the recess are sufficient to accommodate a solid part of the acupressure element, but without close contact between the edge of base of the acupressure elements solid part and the recess walls. This option is the least demanding in production and use, but it has certain limitations in application. In the manufacture of individual medallions in this device, the solid part of the acupressure element can be placed with the support side of the base down in the recess, and then the low-melting material may be placed from above, or the solid part may be immersed with spiked outgrowths down into the previously placed molten low-melting material. If there are many depressions at the same time in the said base, for the manufacture of many medallions at once, their use is possible only by immersion with spiked outgrowths down into the recess filled with molten material. However, in the manufacture of many medallions at once, this version of the device for the manufacture of acupressure element allows to quickly immerse many solid parts of acupressure elements, fixed on the substrate (mat, etc.), due to lack of close contact between the edges of the solid part of the acupressure element and recess walls.

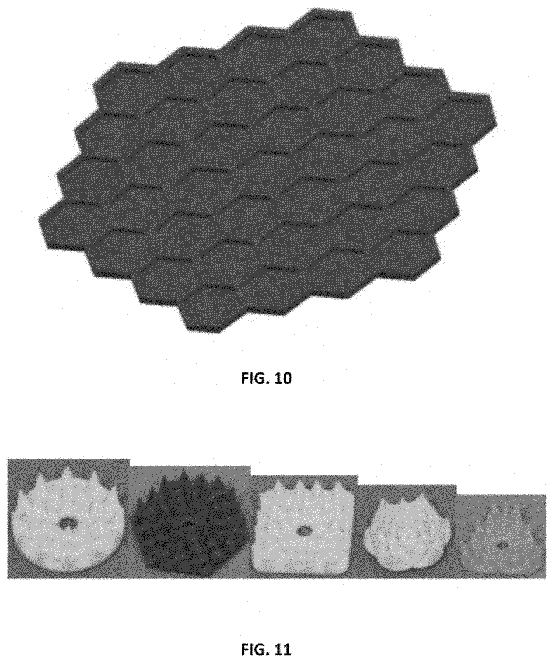

This variant of the device, with “free” cells, as well as all other embodiments of the device according to the invention, can be adapted for use with any shape of applicator elements, or solid parts of the acupressure element (medallion), according to the invention. In particular, FIG. 10 shows a device adapted for use with hexagonal applicator elements. Various embodiments of acceptable applicator elements are illustrated in the image on the FIG. 11 (round, hexagonal, square, triangle, “flower”).

Usually after manufacturing the finished acupressure element is removed from the device for its manufacture, and stored in conditions that depend on the nature of the low-melting material. If the low-melting material is made on the basis of ice, temperatures below 0° C. are required for storage. Low melting material of a different nature may allow storage at room temperature, taking into account the difference between typical room temperatures in different regions and climatic zones of the globe.

But the finished acupressure element can be also stored without removing from the device for its manufacture. The device for the manufacture of acupressure element(s) can be used as packaging for convenient storage and transportation of the finished acupressure element(s). Such a device can even be designed for single use, also playing the role of packaging, including marketing packaging. Or such device together with the acupressure element formed in it can have additional external packaging. In this form, the said device together with the acupressure element comprises the set described in the previous section. In addition to the convenience of storage and transportation, this set according to the invention can perform the function of preventing the leakage of low-melting material in case of non-compliance with the conditions of storage, and melting of this material. This is done either by close contact of the base of the acupressure element with the walls of the cell of the device in the set, or the presence of an outer airtight package, which prevents leakage of low-melting material of the set.

The use of ready-made acupressure elements (medallions) is as follows. Before contact with the skin, the medallions are laid out on the base—a flat surface, spiked outgrowths up. Before that, the substrate (a pad, a mat) can be laid out, made with the means of fixing acupressure elements (medallions) on it, then the medallions are fixed on this substrate before use. In the variant of fixing a number of medallions on one substrate (a mat), the advantage is that this substrate with medallions on it is quickly placed on an acceptable flat surface. The consumer, who is about to become accustomed to the discomfort of acupressure reflexotherapy, then lies down on the medallion or medallions, or provides pressure on selected areas of the skin in another way (for example, pressure by hand or foot, wrapping with a cloth or bandage, or fixed on it by means of special bandages with fasteners (buttons, pins, hooks, sticky fasteners, etc.). If a low-melting material is used which needs to be heated, then, after applying pressure on the medallions, they are heated from the support side, for example, by means of an electric or water heater placed under them or under the substrate to which the medallions are attached.

The procedure of getting used to acupressure reflexotherapy can take any time and depends entirely on the consumer. For example, it can last 5-15 minutes, but it can be interrupted in a minute or two, depending on the general state of the consumer. Gradually, as one gets used to it, the consumer can move from smooth medallions to medallions that do not completely cover the spiked outgrowths, that protrude by 0.5-5 mm.

Immediately before the massage procedure, it is acceptable to warm the skin in a sauna, rubbing with special (including warming) creams (substances) and a simple traditional massage. Such contrast of temperatures in combination with acupressure massage tones up well the receptors of the body, which are connected with all the internal organs of the human body. Conversely, in hot weather, the preferred use of medallions may be the use of ice as a low-melting material, which will also achieve a pleasant consumer's effect.

1. An acupressure element, comprising:
a solid part, comprising a base, having support side and working side, having at least one spiked reflex action outgrowth on the working side, and
a layer of low-melting material capable of melting or softening at a temperature that human skin can withstand at least for 1 minute,
wherein the said layer of low-melting material is made by filling the volume on the working side of the base, between the base and the spiked outgrowth so that said layer forms a surface that is essentially at the same level with a point of spiked outgrowth, or at a level lower than the said point by 0.5-5 mm.

2. The element according to claim 1, wherein a surface of the layer of low-melting material is at the same level as the point of the spiked reflex action outgrowth.

3. The element according to claim 1, wherein a surface of the layer of low-melting material is below the point by 0.5-5 mm.

4. The element according to claim 1, wherein the low-melting material melts at temperatures from 0 to 48° C.

5. The element according to claim 1, wherein the low-melting material is selected from a group consisting of: ice and an aqueous gel, said aqueous gel being selected from a gel based on one of a group consisting of: agarose, agar-agar, carrageenan, alginates and gelatin.

6. The element according to claim 1, wherein the low-melting material is selected from a group consisting of: solid vegetable or animal fat, wax, propolis, vaseline, paraffin, a mixture of paraffin and ozokerite, or a mixture of any or all of these materials.

7. The element according to claim 5, wherein the low-melting material further comprises excipients and/or active ingredients for medicinal, cosmetic or consumer purposes.

8. The element according to claim 1, wherein the low-melting material is comprised of gallium or its low-melting alloy.

9. The element according to claim 1, wherein the base and/or spiked reflex action outgrowth(s) are comprised of material selected from the group consisting of: plastic, wood, metal, rubber, silicone and ceramics.

10. The element according to claim 1, wherein the base has a shape of one of a group consisting of: a square, circle, oval, hexagon, triangle and polygon.

11. The element according to claim 1, wherein the spiked reflex action outgrowth has the shape of one of a group consisting of: a cone, a pyramid with a base having three-, four- and/or more angles, petals or blades with a tapered angle or side, a nail or a needle.

12. The element according to claim 1, wherein the supporting side of the base is comprised of means of fixing or fastening on an underlying surface or a mat.

13. The element according to claim 1, wherein the fastening means are represented by one of a group consisting of: hole, loop, button, pin, hook and/or adhesive clasp.

14-22. (canceled)

23. A device for manufacturing, comprising:
one cell with a bottom, or a plurality of cells with a bottom, connected side by side, and the size and shape of the cell are sufficient to accommodate a solid part of the acupressure element, freely, without contact between the edge of the base of the solid part of the acupressure element and the cell walls
wherein height of the cell is not lower than 5 mm of the height of the spiked reflex action outgrowth(s) over the above mentioned base of the element.

24. The device according to claim 23, wherein said free cells with a bottom have a height that is lower than 0.5-5 mm of the sum of the height of the spiked reflex action outgrowth and the thickness of the base of the acupressure element.

25. The device according to claim 23, wherein said free cells with a bottom have a height equal to or greater than the sum of the height of the spiked outgrowth of the reflex action and the thickness of the base of the acupressure element.

26. The device according to claim 23, being comprised of a material selected from a group consisting of: rubber, silicone, metal, plastic, cardboard, paper, foil, paper, laminated with foil or plastic, and wood.

27. A set, comprising:
an element according to claim 1; and
a device comprising: one cell with a bottom, or a plurality of cells with a bottom, connected side by side, and the size and shape of the cell are sufficient to accommodate a solid part of the acupressure element, freely, without contact between the edge of the base of the solid part of the acupressure element and the cell walls,
wherein height of the cell is not lower than 5 mm of the height of the spiked reflex action outgrowth(s) over the above mentioned base of the element.

28. The set, according to claim 27, wherein the element is placed in the device.

29. The set according to claim 27, further comprising: a leakage-proof package.

30-39. (canceled)
